(19) United States
(12) Patent Application Publication
(10) Pub. No.: US 2024/0200024 A1
(43) Pub. Date: Jun. 20, 2024

(54) MICROPARTICLES MADE OF PLANT PROTEIN EXTRACTS AND USES THEREOF AS CELL CARRIERS FOR THE PREPARATION OF FOOD PRODUCTS

(71) Applicant: Technion Research & Development Foundation Limited, Haifa (IL)

(72) Inventors: Marcelle MACHLUF, Haifa (IL); Ayelet FISHMAN, Haifa (IL); Maya DAVIDOVICH-PINHAS, Haifa (IL); Limor BARUCH, Haifa (IL); Anton ZERNOV, Haifa (IL); Jovana GLUSAC, Haifa (IL)

(73) Assignee: Technion Research & Development Foundation Limited, Haifa (IL)

(21) Appl. No.: 18/586,768

(22) Filed: Feb. 26, 2024

Related U.S. Application Data
(63) Continuation of application No. PCT/IL2022/050929, filed on Aug. 25, 2022.
(60) Provisional application No. 63/236,885, filed on Aug. 25, 2021.

(51) Int. Cl. C12N 5/00; A23J 3/14; C12N 9/10

(52) U.S. Cl. CPC C12N 5/0075; A23J 3/14; C12N 9/1044; C12N 2533/50; C12N 2533/72; C12N 2533/74; C12N 2537/10

(57) ABSTRACT

Compositions-of-matter containing a plurality of edible microstructures (e.g., microparticles) made of a plant protein extract, composites containing such compositions-of-matter and cells adhered to the microstructures, and processes of preparing same, are provided. Also provided are food products containing these composites and methods of preparing same.

### RELATED APPLICATIONS

This application is a Continuation of PCT Patent Application No. PCT/IL2022/050929 having International filing date of Aug. 25, 2022, which claims the benefit of priority under 35 USC § 119(e) of U.S. Provisional Patent Application No. 63/236,885 filed on Aug. 25, 2021. The contents of the above applications are all incorporated by reference as if fully set forth herein in their entirety.

### FIELD AND BACKGROUND OF THE INVENTION

The present invention, in some embodiments thereof, relate to edible products, and, more specifically, but not exclusively, to edible microstructures which are usable as cell carriers, to edible composites made of these microstructures and cells, and to food products made of these composites.

Cultured meat involves the application of tissue engineering practices to produce muscle from isolated animal cells for consumption as food. A wide consensus exists regarding the urgent necessity to develop viable solutions that will facilitate the introduction of commercial cultured meat products, which is motivated not only by the concern for animal welfare but, most importantly, by the need to provide sustainable food resources to the world's growing population. Hence, cultured meat holds a great promise of facing this shortage in food resources without compromising safety, nutritional values, or environmental concerns. Nevertheless, the realization of such solutions still faces major challenges, primarily technological ones such as the development of proper polymeric cell carriers and scaffolds relevant to cultured meat applications that will enable the large-scale production of cultured meat in a commercially-viable manner.³ In addition, the development of trans-fat-free fat substitutes that will resemble the sensorial and nutritional attributes of animal-derived fat is needed for the design of high-quality cultured meat products.

Additional background art includes:
WO 2015/038988;
U.S. Patent Application No. 20150150283 and 20200140810;
Bodiou et al. Front. Nutr., 20 Feb. 2020 www (dot)doi(dot)org/10(dot)3389/fnut(dot)2020(dot)00010.
Stout 10(dot)1016/j(dot)biomaterials(dot)2022(dot)121659.

### SUMMARY OF THE INVENTION

According to an aspect of some embodiments of the present invention there is provided a composition-of-matter comprising a plurality of edible microparticles at least a portion of the edible microparticles comprises a plant protein.

According to some of any of the embodiments described herein, the plant protein is in a plant protein extract.

According to some of any of the embodiments described herein, the plant of the plant protein is a legume.

According to some of any of the embodiments described herein, the legume is selected from the group consisting of a chickpea, lentil, pea, bean and soybean.

According to some of any of the embodiments described herein, the legume is chickpea.

According to some of any of the embodiments described herein, at least a portion of the microparticles are essentially irregular in shape.

According to some of any of the embodiments described herein, at least a portion of the microparticles are essentially spherical.

According to some of any of the embodiments described herein, the microparticles comprise an oleogel core.

According to some of any of the embodiments described herein, the microparticles comprise the oleogel core and a continuous or non-continuous coating comprising the plant protein.

According to some of any of the embodiments described herein, the oleogel core comprises an edible oil and an edible structuring agent.

According to some of any of the embodiments described herein, the edible structuring agent comprises glycerol monostearate.

According to some of any of the embodiments described herein, the coating is about 10-40 μm thick.

According to some of any of the embodiments described herein, the plant protein is cross-linked using an enzyme.

According to some of any of the embodiments described herein, the composition-of-matter further comprises an edible polymer.

According to some of any of the embodiments described herein, the edible polymer is selected from the group consisting of a lipid, a protein and a polysaccharide.

According to some of any of the embodiments described herein, the edible polymer is selected from the group consisting of an alginate, a chitosan and a dextrin.

According to some of any of the embodiments described herein, the edible polymer is cross-linked, and wherein a cross-linking agent for cross linking the edible polymer is edible.

According to some of any of the embodiments described herein, the edible polymer is alginate and the cross-linking agent for cross linking the edible polymer is Ca⁺².

According to some of any of the embodiments described herein, the plant protein is cross-linked.

According to some of any of the embodiments described herein, the cross-linked plant protein extract is cross-linked using a chemical or using an enzyme.

According to some of any of the embodiments described herein, the enzyme is transglutaminase (TG).

According to some of any of the embodiments described herein, the chemical is an organic acid.

According to some of any of the embodiments described herein, the organic acid is citric acid.

According to some of any of the embodiments described herein, the chemical is epigallocatechin gallate (EGCG).

According to some of any of the embodiments described herein, at least one of the plant protein and edible polymer is cross-linked.

According to some of any of the embodiments described herein, the microparticles consist essentially of the plant protein extract.

According to some of any of the embodiments described herein, the microparticles are produced by electrospraying an aqueous solution of the plant protein extract into an alcohol solution.

According to yet a further embodiment of the present invention, the drying and final coating are carried out in one step in a fluidized bed apparatus.

According to some of any of the embodiments described herein, the aqueous solution of the plant protein comprises a volatile water-miscible organic solvent.

According to some of any of the embodiments described herein, the organic solvent is ethanol.

According to some of any of the embodiments described herein, the composition-of-matter is lyophilized.

According to some of any of the embodiments described herein, the plant protein comprises denatured protein.

According to some of any of the embodiments described herein, an average size of the microparticles is 50-600 μm.

According to some of any of the embodiments described herein, an average zeta potential of the microparticles is −30 to −70 mV when comprising an oleogel core.

According to an aspect of some embodiments of the present invention there is provided a method of producing the edible particles, the method comprising particulating a composition comprising an edible plant protein and optionally an edible polymer under conditions which allow formation of edible microparticles which support proliferation of cells cultured thereon.

According to some of any of the embodiments described herein, the particulating comprises cross linking at least one of the edible plant protein and the edible polymer.

According to some of any of the embodiments described herein, the composition further comprises an edible oil and an edible structuring agent.

According to an aspect of some embodiments of the present invention there is provided a composition-of-matter comprising microparticles produced according to the method as described herein.

According to an aspect of some embodiments of the present invention there is provided a composite comprising the composition-of-matter as described herein and cells adhered to the microparticles.

According to some of any of the embodiments described herein, the cells comprise terminally differentiated cells.

According to some of any of the embodiments described herein, the cells are selected from the group consisting of muscle cells, fibroblasts, adipose cells and blood cells.

According to some of any of the embodiments described herein, the cells are stem cells or progenitor cells.

According to some of any of the embodiments described herein, the cells are mesenchymal stem cells.

According to some of any of the embodiments described herein, the cells are of an animal origin.

According to an aspect of some embodiments of the present invention there is provided a food comprising the composition of composite as described herein.

According to an aspect of some embodiments of the present invention there is provided a method of culturing, comprising culturing the composite as described herein under conditions which allow differentiation and/or proliferation of the cells on the microparticles.

According to some of any of the embodiments described herein, the cells are from a domesticated animal.

According to some of any of the embodiments described herein, the method further comprises retrieving the cells without discarding the microparticles.

According to an aspect of some embodiments of the present invention there is provided a method of producing food, the method comprising fabricating food comprising the composite or composition as described herein.

According to some of any of the embodiments described herein, the fabricating comprises molding, extruding, electrospinning and/or printing.

According to an aspect of some embodiments of the present invention there is provided a composition-of-matter comprising a plurality of edible microstructures, wherein at least a portion of the edible microstructures comprises a plant protein extract.

According to some of any of the embodiments described herein, the plant of the plant protein extract is a legume.

According to some of any of the embodiments described herein, the legume is selected from the group consisting of a chickpea, lentil, pea, bean and soybean.

According to some of any of the embodiments described herein, the legume is chickpea.

According to some of any of the embodiments described herein, the microstructures comprise microparticles, such that at least 50%, or at least 70%, or at least 80%, or at least 90%, or essentially all, of the microstructures are microparticles.

An average size of the microparticles depends of their composition and/or method of preparation, and can range from 1 micron to 800-900 microns.

According to some of any of the embodiments described herein, at least a portion of the microparticles are essentially irregular in shape.

According to some of any of the embodiments described herein, at least a portion of the microparticles are essentially spherical.

According to some of any of the embodiments described herein, the microparticles comprise an oleogel core.

According to some of any of the embodiments described herein, the microparticles comprise the oleogel core and a continuous or non-continuous coating comprising the plant protein extract.

According to some of any of the embodiments described herein, the oleogel core comprises an edible oil (e.g., canola oil) and an edible structuring agent (a gel-forming agent or a gelating agent) such as glycerol monostearate, ethyl cellulose and like cellulose-containing gelating agents, edible waxes, free fatty acids, and more.

According to some of any of the embodiments described herein, the plant protein extract is cross-linked (e.g., enzymatically cross-linked, for example, using transglutaminase, or otherwise a chemically-cross linked plant extract protein).

Exemplary microparticles that comprise an oleogel are described in Example 4.

According to some of any of the embodiments described herein, the composition-of-matter further comprises an edible polymer (e.g., alginate).

According to some of these embodiments, the edible polymer is cross-linked.

According to some of these embodiments, the cross-linking agent is edible, and preferably the cross-linking process does not involve agents that are not biocompatible.

In exemplary embodiments, the cross-linked polymer is a cross-linked alginate, for example, Ca²⁺-crosslinked alginate.

According to some of any of the embodiments described herein, the plant protein extract is cross-linked. According to some of these embodiments, the plant protein extract is cross-linked by edible or biocompatible cross-linking agents and cross-linking conditions.

According to some of any of the embodiments described herein, the cross-linked plant protein extract is cross-linked enzymatically (e.g., using transglutaminase).

According to some of any of the embodiments described herein, the cross-linked plant protein extract is cross-linked chemically (e.g., using an edible organic acid such as citric acid or using an edible cross-linking agent such as, for example, epigallocatechin gallate (EGCG).

According to some of any of the embodiments described herein, the plant protein extract is cross-linked (e.g., enzymatically and/or chemically), and wherein the composition-of-matter further comprises an edible cross-linked polymer (e.g., cross-linked alginate, for example, Ca²⁺-crosslinked alginate).

Exemplary compositions-of-matter that further comprise an edible polymer are described in Example 2. In some embodiments, an average size of the microparticles ranges from 100 to 800, or from 200 to 700, microns. Depending on the preparation method and components, such microparticles can be spherical or irregular in shape.

According to some of any of the embodiments described herein, the composition-of-matter or the microparticles consist essentially of the plant protein extract, or are devoid of an edible polymer or of a cross-linked edible polymer.

In some of these embodiments, the microparticles are prepared by electrospraying an aqueous solution of the plant protein extract (preferably containing a water-miscible organic solvent which is volatile) into an alcohol (e.g., ethanol) solution, as described, for example, in Example 3.

According to some of any of the embodiments described herein, the microstructures or the composition-of-matter are/is lyophilized.

According to some of any of the embodiments described herein, the plant protein extract comprises denatured protein.

According to an aspect of some embodiments of the present invention there is provided a composite comprising the composition-of-matter as described herein and cells adhered to the microparticles.

According to some of any of the embodiments described herein, the cells are selected from the group consisting of muscle cells, fibroblasts, adipose cells, blood cells and more.

According to some of any of the embodiments described herein, the cells are stem cells or progenitor cells.

According to some of any of the embodiments described herein, the cells are mesenchymal stem cells.

According to some of any of the embodiments described herein, the cells are of an animal/fish/avian origin.

According to an aspect of some embodiments of the present invention there is provided a food (a food product) comprising the composite as described herein.

According to an aspect of some embodiments of the present invention there is provided a method of culturing, comprising culturing the composite under conditions which allow differentiation and/or proliferation of the cells on the microparticles.

According to some of any of the embodiments described herein, the method further comprises retrieving the cells without discarding the microparticles.

According to an aspect of some embodiments of the present invention there is provided a method of producing food (a food product), the method comprising facricating food comprising the composite as described herein.

According to some of any of the embodiments described herein, the fabricating comprises molding, extruding, electrospinning and/or printing.

Some embodiments of the present invention focus on the most important aspects of mammalian cell culture: The development of cell carriers based on chickpea protein extract (CPE) for the cultivation, expansion, and structure design of mammalian cell cultures.

Some embodiments of the present invention relate to CPE-oleogel fat substitutes of different types, such as protein-coated oleogel microparticles to be used as fat substitute and as cell carriers.

Some embodiments of the present invention relate to compositions-of-matter that comprise edible microstructures such as microparticles, that comprise a plant protein extract (e.g., chickpea protein extract), to processes of preparing same, to composites containing same and cells adhered to the microstructures, to use of such composites for preparing food products and to food products containing such composites.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows a culture of bMSC on CPE-coated non-adherent tissue culture (TC) plates, compared to regular TC plates, non-adherent TC plates, and gelatin-coated TC plates. The cultured cells were imaged under bright field microscopy (inserts) and further stained with DAPI and phalloidin to visualize cell morphology (large images).

FIG. 2 shows expression of CD29 and CD44 by bMSCs cultured on CPE-coated non-adherent TC plates, compared to regular TC plates, non-adherent TC plates, and gelatin-coated TC plates.

FIG. 3 shows the effect of CPE on the viability of sheep fibroblasts and bMSCs through 4 days of culturing. Cells were seeded at 10,000 cells/cm² or 2,500 cells/cm².

FIG. 4 shows chickpea-alginate cell carriers according to some embodiments of the invention.

FIG. 5 shows chickpea-alginate cell carriers produced using TG crosslinking and citric acid treatment according to some embodiments of the invention. Images show the microcarriers following TG treatment in different buffers (first row) and subsequent citric acid treatment (second row). Third row: attachment of bMSC to the cell carriers (Green/bright spots: living cells).

FIG. 6 shows chickpea-alginate cell carriers produced by direct crosslinking with citric acid. Seeded C2C12 cells are stained with FDA (green/bright spots or pathces).

FIG. 7 shows 100% chickpea protein cell carriers. bMSC seeded on the carriers are stained with FDA (green/bright spots or pathces).

FIGS. 8A-G show a general scheme for oleogel production and results of characterization. (A) Oleogel production scheme based on o/w emulsion template. (B) Confocal laser scanning microscopy (CLSM) images of o/w emulsion are used as a template for oleogel production. NR— refers to Nile red fluorescence signal (for fat); NB— refers to Nile blue fluorescence signal (for protein), while NR+NB shows a superposition of both signals. The scale bar of 14 μm for all CLSM images, except that lower microscopy images have different magnification and scale bar (4 μm). (C) Polarized light microscopy images of oleogel with different magnification. (D) Thermal gravimetric analysis (TGA) curves represent the degradation behaviour over the temperature of the oleogel and beef fat. (E) Differential scanning calorimetry (DSC) thermogram (10° C. min-) of oleogel and beef fat. (f) Hardness (N) values of raw and cooked oleogels and beef fat. (G) Elemental analysis (C, H, N, and S) of oleogel and beef fat.

FIGS. 9A-D show size distribution of oleogel particles formulated using 0.5% (A), 1% (B), 1.5% (C), or 2% (D) chickpea protein 1 day following production and along 4 weeks of storage at 4° C.

FIG. 10 shows the mean (±SD) zeta potential of chickpea o/w emulsions with different CP concentration (0.1, 0.5, 1, 1.5 and 2%) and 50 U/g TG over 1-month storage at 4° C.

FIGS. 11A-H show SEM micrographs of lyophilized oleogel microparticles formulated using 0.5% (A-B), 1% (C-D), 1.5% (E-F), or 2% (G-H) protein. Images were captured using SEM PHENOM. Emulsions were 2 days old before lyophilization. Red arrows show the thickness of the protein layer.

FIG. 12 shows size distribution of oleogel particles before lyophilization (full line) and redispersion of lyophilized emulsions (dashed line).

### DESCRIPTION OF SPECIFIC EMBODIMENTS OF THE INVENTION

The present invention, in some embodiments thereof, relate to edible products, and, more specifically, but not exclusively, to edible microstructures which are usable as cell carriers, to edible composites made of these microstructures and cells, and to food products made of these composites.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The cultured meat industry relies on large scale production of animal cells which form an edible product. These usually include connective tissue cells, muscle cells and fat cells. The latter two are adherent and their large-scale production in suspension is a limiting factor in the development of this industry with the ultimate goal: the production of affordable commodities. Tissue culture carriers (also known as “cell carriers” “microcarriers”) provide physical and biochemical cues for the cells to adhere, proliferate and differentiate into the necessary cell types and if fabricated from an edible material, allow spatial heterogeneity in the final product that will resemble the natural meat structure and texture. The carrier should ideally aid in some desirable organoleptic properties, another consideration is whether the carrier should be part of the final product and therefore made edible, food-grade and nutritious, or if it should be designed to be removable. The main purpose of the carrier is to facilitate necessary muscles, fat and connective tissue development. However, to date these needs have not been met.

Whilst conceiving and reducing to practice embodiments of the present invention, the present inventors have produced hydrogel microparticles, which are edible, nutritious, stable and support proliferation and differentiation of cells cultured thereon, as exemplified on mesenchymal stem cells. These particles are composed of plant proteins and especially legumes which are of high nutritional value. Formulations thereof to comprise an oleogel core render them especially useful to combine with other food components for improving the organoleptic property of foods.

Thus, according to an aspect of the invention, there is provided a composition-of-matter comprising a plurality of edible microparticles at least a portion of said edible microparticles comprises a plant protein.

As used herein “edible” refers to a composition which is safe for human or animal eating. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

According to a specific embodiment, the composition is animal-free, i.e., does not comprise any substance which is isolated from an animal body.

As used herein “food grade” refers to a substance which is either safe for human consumption or confirmed to come into direct contact with food products.

According to some embodiments of the invention, the materials used in the context of the invention are of “food grade” classification.

Such substances are also referred to herein and in the art as “food contact substances” or “food contact materials”.

The phrase “food contact substance” or FCS, is used herein to describe substances that are generally safe for human consumption by virtue of being generally recognized as safe (GRAS) or by passing standard safety tests, and thus qualify for use as a component of materials used in manufacturing, packing, packaging, transporting, or holding food, in the same manner it is meant in the guideline and regulation of worldwide food administration authorities, such as, for example, the U.S. Food and Drug Administration (FDA), Center for Food Safety and Applied Nutrition (CFSAN), the Office of food Additive Safety.

The phrase “generally recognized as safe” or GRAS, as used herein, is meant in the same manner which is defined, for example, under sections 201(s) and 409 of the U.S. FD&C Act. The U.S. law states that any substance that intentionally contacts food or added to food is a food additive, that is subject to premarket review and approval by FDA, unless the substance is generally recognized, among qualified experts, as having been adequately shown to be safe under the conditions of its intended use, or unless the use of the substance is otherwise excluded from the definition of a food additive. GRAS substances are distinguished from food additives by the type of information that supports the GRAS determination, that it is publicly available and generally accepted by the scientific community, but should be the same quantity and quality of information that would support the safety of a food additive. Since the qualification to an FCS or GRAS category can be obtained through a process of applying, testing and qualifying to the requirements of the various official food and drug authorities, the present embodiments are meant to encompass all relevant substances and their derivatives which are to become FCSs and GRAS in the future, as well as those which already qualify as FCSs and GRAS.

As used herein “plant protein” refers to a protein of a plant or a functional homolog of same (naturally occurring or synthetic).

According to a specific embodiment, the plant protein is leguminous. The proteins of legumes are mainly storage proteins belonging to the groups of albumins, globulins and glutelins, with the salt-soluble globulins constituting the main prorins found in the seeds. There are also a number of proteins, other than the water-soluble storage proteins, mainly enzymes, enzyme inhibitors and lectins which form a part of a protein extract. According to an embodiment of the invention, this latter group is present in an amount which is lower than that naturally present in the whole protein extract of the plant.

As used herein “plant protein extract” refers to concentrates or isolates with a protein content of about 70-98% respectively. According to some embodiments, techniques useful for plant (e.g., legume) exploitation in preparing rich-in protein materials include, but are not limited to, air classification and wet protein extraction methods (Tiwari et al. 2011 Pulse Foods: Processing, quality and nutraceutical applications. London, Academic Press p. 483).

Methods of protein extraction are further described hereinbelow and in the Examples section. See for instance, Glusac J, Isaschar-Ovdat S, Fishman A. Transglutaminase modifies the physical stability and digestibility of chickpea protein-stabilized oil-in-water emulsions. Food Chem. 2020; 315:126301.

According to another embodiment, the protein extract is commercially available. Such include, but are not limited to, ChickP, InnovoPro Green Boy.

As used herein “legume” refers to any plant from the Fabaceae family. A pulse is the edible seed from a legume plant. Pulses include beans, lentils, and peas.

According to a specific embodiment, the legume belongs to a type selected from the group consisting beans, peas, peanuts, lentils, and lupins.

According to a specific embodiment, the protein is from a legumninous plant e.g., lentils, lupin, peas, cowpeas, fava beans, chickpeas, and soybean.

Lupin belong to the Genisteae family, Fabaceae or Leguminosae and more than 400 species are known, from which only four are of agronomic interest: Lupinus albus (white lupin), Lupinus angustifolius (narrow leaf or blue lupin), Lupinus luteus (yellow lupin) and Lupinus mutabilis (Andean lupin). Lupin generally contains about twice the amount of proteins found in those legumes that are commonly consumed by humans. Globulins (α-conglutin or 11S-like protein, β-conglutin or 7S-like protein, and 7-conglutin) are the main storage proteins (80-90%) in lupins while prolamines and glutelins are detected in small amounts similar to those reported in most legume seeds. Lupin is source of sulphur-containing amino acids and arginine, and has a good balance of essential amino acids with a high degree of digestibility.

The oil content of lupin may range from 1 to 17%, with a high variation in fatty acid composition. The dominating fatty acids are oleic and linoleic acid. The seeds contain low amounts of starch (0 to 5%), and structural polysaccharides such as hemicellulose. The primary cell walls of cotyledons are composed of arabinogalactans, arabinans, rhamnogalacturosan, galactoxyloglucans and galactans. Mature seeds of yellow lupin accumulate stachyose, verbascose and raffinose, amounting 11% of dry mass, and sucrose (1.5%). Lupin seeds contain about 1.5 to 3.5% of sucrose, a relatively high amount of stachyose (6.0 to 7.5%). The raffinose and verbascose content are about 0.5 to 0.9%, and 0.3 to 0.8% respectively.

Lupin seeds, like other legumes are good sources of vitamins and phenolic compounds. Carotenoids and tocopherols are present in such legume extracts, with the former being mainly responsible for the color of the oil fraction.

Peas are a genus of the family Fabaceae. It contains one to five species, depending on taxonomic interpretation. Pisum sativum (the field or garden pea), is domesticated and is a major human food crop. Like other legume seeds, pea is rich in protein (18-30%) and also contain vitamins, minerals and dietary fiber [. Pea seed storage proteins are composed mainly of legumin (11S), vicillin (7S) and albumins (2S) and the majority of pea protein isolates contain globular 11S and 7S]. The ratio of legumin to vicilin in pea ranges from 0.2 to 1.5.

Beans are one of most consumed legume worldwide. Beans are reported to contain 17.96-23.62% proteins, 1.27-3.62% fat, 2.86-5.00% ash and 56.53-61.56% carbohydrates. They have a balanced amino acid composition while they are low in sulfur-containing amino acids (methionine and tryptophan) as is the case with other pulses. They contain vitamins. The storage proteins of beans are vicilin and legumin. Vicilin is a 7S globulin and is often referred to as phaseolin. It is comprised of 3-5 subunits and represents 50% of the total protein content. Legumin is an 11-125, globulin, comprised of acid and basic subunits, and usually sediments with vicilin as a single.

Soybeans are widely used in the food industry because of its high protein and oil contents. Soybeans can be classified into oil bean and food bean according to its end uses. Oil soybean, i.e., commodity bean, is the primary source of vegetable oil and soy protein products, such as defatted soy flour and soy protein concentrate; food bean, i.e. specialty bean, is either consumed directly or processed into various soy products. The composition of soybeans may vary somewhat according to variety and growing conditions. Through plant breeding it has been possible to obtain protein levels between 40% and 45%, and lipid levels between 18 and 20%. Usually, an increase of 1% in protein content is accompanied by a decrease of 0.5% in oil.

The four major fractions of soybeans protein are known as 2S, 7S, 11S and 15S [15]. The 11S and 7S fractions constitute about 70% of the total protein in soybeans. The ratio 11S/7S is a varietal characteristic and may vary from 0.5 to 3. The 2S fraction consists of low molecular weight polypeptides (in the range of 8000 to 20000 daltons) and comprises the soybean trypsin inhibitors. The 7S fraction is highly heterogeneous. Its principal component is beta-conglycinin, a sugar containing globulin with a molecular weight in the order of 150000. The fraction also comprises enzymes (beta-amylase and lipoxygenase) and hemagglutinins. The 11S fraction consists of glycinin, the principal protein of soybeans. Glycinin has a molecular weight of 320000-350000 and is built of 12 subunits, associated through hydrogen bonding and disulfide bonds. The ability of soy proteins to undergo association-dissociation reactions under known conditions, is related to their functional properties and particularly to their texturization. The 15S protein is probably a dimer of glycinin. Conglycinin and glycinin are storage proteins and they are found in the protein bodies within the cells of the cotyledons. The limiting amino acids are the ones containing sulphur (methionine and cystine). Soybean protein is exceptionally rich in lysine and can serve as a valuable supplement to cereal foods where lysine is a limiting factor.

Chickpea (Cicer arietinum) is an annual legume of the family Fabaceae, subfamily Faboideae. Its different types are variously known as gram or Bengal gram, garbanzo or garbanzo bean, or Egyptian pea. Chickpea seeds are high in protein. Thus, protein represents an important component of chickpea seeds. Indeed, chickpeas have a high content of protein, typically ˜20%-25% and, according to the Osborne fractions classification, the principal proteins in chickpeas are albumin, globulin, prolamin, andglutelin, representing 8%-12%, 53%-60%, 3%-7%, and 19%-25% of total protein, respectively. Some variation can be seen in the protein content of the two seed types, “desi” and “kabuli” which may arise from differences in the environment in which they are grown, agronomic techniques used, or storage conditions. Differences in amino acid patterns between “desi” and “kabuli” chickpeas have been reported, for example methionine content was 1.4 and 1.1 g/100 g of protein for “desi” and “kabuli” flours, respectively, while more significant differences were observed in leucine, lysine and serine content between the two seeds (4.2 and 2.5, 7.2 and 7.6, 5.4 and 7.3 g/100 g of protein, respectively).

According to a specific embodiment, the legume is chickpea.

Methods of producing protein extracts from plants are well known in the art (Kumar et al. Food Hydrocolloids Volume 115, June 2021, 106595).

According to a specific embodiment, the plant is a legume and the protein is isolated/extracted from pulse seeds.

As mentioned, the major proteins found in legumes are globulins and albumins. Albumins are water soluble and comprise enzymatic proteins, protease inhibitors, amylase inhibitors and lectins and have molecular masses (MM) ranging between 5000 and 80,000 Da. Globulins represent roughly 70% of legume seed proteins and consist primarily of the 7S, 11S and 15S proteins. Molecular weights of these proteins range from 8 KDa to 600 KDa. These proteins generally have a minimum solubility at pH values between 4 and 5 (isoelectric point). By manipulating the solubility of the proteins and using filtration techniques that take advantage of their hydrodynamic properties, protein concentrates and isolates with varying purity and functionality can be obtained, according to some embodiments of the invention.

As mentioned, protein extraction processes generally used are (1) pin-milling plus air-classification which when applied to starch-rich legume seeds (peas, beans), results in concentrates (defined as having protein contents of 60-75%), and (2) wet processes (e. g. alkaline extraction/isoelectric precipitation, acid extraction) which produce isolates (defined as having protein contents of 90% to 95%). By air-classification, concentrates having 68% and 65% protein can be obtained, respectively, from bean (31% protein) and pea (21% protein). Isolates, prepared by extraction of the flour proteins with alkaline solution followed by acid precipitation, have a protein content generally between 90% and 96% and a protein recovery yield varying between 60% and 65%.

Air classification and pin milling are generally used to fractionate legumes into a light or fine fraction (protein concentrate) and a heavy or coarse fraction (starch concentrate). Using this method, whole or dehulled seeds of legumes are pin—milled and then yielded flours are fractionated into “protein” and “starch” concentrates using air classifier. The purity of the protein fraction obtained using this process is, however, low (38-65%) and further processing may be required.

Alkaline extraction and acidic (isoelectric) precipitation is one of the commonly applied methods for protein isolates. The basis of such method lies on the application of different solubility and precipitation profiles of proteins. Higher solubility is observed at the alkaline and acid pH range whereas lowest solubility occurs at the isoelectric point (around pH 4-5). This phenomenon provides the basis for many protein isolation techniques as acid, alkaline extraction and isoelectric precipitation. During the process of alkaline extraction and acidic precipitation of protein, the raw material is usually subjected to an alkaline pH level between pH 8 and 11 where the protein is found to be the most soluble. The pH of the soluble protein fraction is then adjusted between pH 4 and 5, where the isoelectric points of most of the vegetable proteins lie.

Legume proteins can be produced using acid extraction or may also be directly extracted with water without the subsequent acid precipitation step. The principle of acid extraction is similar to that of alkaline extraction except that the initial protein extraction is conducted under acidic conditions.

Solubility of pulse proteins is also high under very acidic conditions (i. e., pH<4). Authors studying the direct acidification (pH 4.4-4.6) of the supernatant from starch extraction of pin-milled faba beans and peas obtained protein contents of 91.2% and 91.9% for faba bean and pea, respectively.

The salt extraction process, sometimes also referred to as micellization is based on the salting-in and salting-out phenomenon of food proteins. In this process, after extraction of protein using an appropriate salt solution at desired ionic strength, the solution is diluted, inducing protein precipitation that can then be recovered by centrifugation or filtration, followed by drying.

Another technique which provides high yield protein isolates with improved functionality is membrane separation. This technique is also useful for depleting the isolates from anti-nutritional compounds (such as lectins and proteinases).

Protein isolate can be produced using alkaline extraction and a novel ultrafiltration/diafiltration (UF/DF) process. Membrane separation is a frequently used alternative to isoelectric precipitation. In this process, the supernatant obtained either after alkaline or acid extraction is subjected to ultrafiltration or ultrafitration/diafiltration to concentrate the proteins. Ultrafiltration (UF) is a pressure-driven membrane process and is one of the most widely used forms of Membrane-based Tangential Flow Filtration (TFF) for proteins separation. Depending on the protein to be retained, membrane nominal molecular weight limits in the range of 1 kD to 1000 kD are used. Diafiltration (DF) is a TFF process that can be performed in combination with ultrafiltration to enhance either product yield or purity.

Additional details for protein extraction from legumes can be found in Boye J., Zare F., Pletch A. Pulse proteins: processing, characterization, functional properties and applications in food and feed/Food Res. Int. 2010. Vol. 43. N 2. P. 414-431. www(dot)dx(dot)doi(dot)org/10(dot)1016/j(dot)foodres(dot)2009(dot)09(dot)003; Gueguen J. Legume seed protein extraction, processing, and end product characteristics/Plant Foods for Human Nutrition. 1983. Vol. 32. N 3-4. P. 267-303. www(dot)dx(dot)doi(dot)org/10(dot)1007/BF01091191; Boye J. I., Aksay S., Roufik S., Ribéreau S., Mondor M., Farnworth E., Rajamohamed S. H. Comparison of the functional properties of pea, chickpea and lentil protein concentrates processed using ultrafiltration and isoelectric precipitation techniques/Food Research International. 2010. Vol. 43. N 2. P. 537-546. www(dot)dx(dot)doi(dot)org/10(dot)1016/j(dot)foodres(dot)2009(dot)07(dot)021; Jayasena V., Chih H. J., Nasar-Abbas S. M. Efficient isolation of lupin protein/Food Australia. 2011. Vol. 63. N 7. P. 306-309; Tyler R. T., Youngs C. G., Sosulski F. W. Air classification of legume. I. Separation efficiency, yield and composition of the starch and protein fractions/Cereal Chemistry. 1981. Vol. 58. N 2. P. 144-148; and Fuhrmeister H., Meuser F. Impact of processing on functional properties of protein products from wrinkled peas/J. Food Eng. 2003. Vol. 56. N 2-3. P. 119-129. www(dot)dx(dot)doi(dot)org/10(dot)1016/S0260-8774(02)00241-8, each of which is incorporated herein by reference in its entirety.

According to a specific embodiment, the plant protein comprises denatured protein.

As used herein “denatured protein” refers to a protein in which the secondary, tertiary, and/or quaternary structures of the protein have been altered, typically by heating though other means are also contemplated hereon. This is done to alter biochemical properties of the protein e.g., solubility or cell-biomaterial interactions.

As used herein the term “microparticles” interchangeable with “particles” and “microcarriers” or “carriers” refers to microstructures which are distinguishable from tubes, fibers and sheets, such as described below.

According to a specific embodiment, the particles comprises pores.

According to a specific embodiment, the pores of the particles are of an average diameter of 1-10 μm, e.g., 1-5 μm.

The average size of the microparticles allows growth of cells on the particles in suspension (as opposed to a scaffold in which cells are grown under two dimensional conditions in static cultures).

According to a specific embodiment, the microparticles are essentially irregular in shape.

According to a specific embodiment, at least a portion of said microparticles are essentially spherical.

The particles of this aspect of the present invention are typically round (see Figures), and more specifically substantially spherical, such as, spherical, oval, semi-spherical, hemispherical, an irregular sphere with flattened sections or concave or convex sections, semi-oval, an irregular oval with flattened sections or concave or convex sections. According to a specific embodiment, the average size of at least one dimension (e.g., a diameter or length) of the particles in a preparation is 50-600 μm, e.g., 100-600 μm, 150-600 μm, 200-600 μm, 250-600 μm, 300-600 μm, 350-600 μm, 400-600 μm, 450-600 μm, 50-550 μm, 50-500 μm, 50-450 μm, 50-400 μm, 50-350 μm, 50-300 μm, 100-500 μm, 200-500 μm, 300-500 m.

As used herein, the term “particle size” refers to the particle size as determined, for example, by microscope image analysis, and laser diffraction analyzer.

As used herein “at least a portion” refers to the predominant part of the particles in a preparation, e.g., at least about 60%, 70%, 80%, 90%, 95%, 99% or even 100%.

According to a specific embodiment, the particles are solid.

According to a specific embodiment, the particles are layered.

According to a specific embodiment, the particles comprise a core.

According to a specific embodiment, the particles comprise an oleogel core.

According to a specific embodiment, the microparticles comprise said oleogel core and a continuous or non-continuous coating comprising said plant protein.

By “continuous coating” it is meant a continuous layer or film that fully or partially envelopes the core.

By “non-continuous coating” it is meant that the coating is of discrete portions of the core.

The term “oleogel” describes a three-dimensional network containing at least 20%, typically at least 50%, or at least 80%, and up to about 99.99% (by mass) of an oily substance as the continuous liquid phase. An oleogel can be regarded as a material which is mostly oil, yet behaves like a solid or semi-solid due to a three-dimensional solid-like network within the liquid dispersing medium. The three-dimensional network is typically formed by a structuring agent, which is also referred to herein and in the art as an oleogel-forming agent or gel-forming agent or as a gelator or oleogelator. According to a specific embodiment, the oleogel core is made of an edible oil as the continuous oily phase and an edible structuring agent that forms, typically self-assembled, to form the three-dimensional network.

Examples of edible plant oils which can be used for forming an oleogel core in accordance with some embodiments of the invention include, but are not limited to those listed in Tables 1 and 2 below (adapted from Zhou et al. Front. Plant Sci., 28 Aug. 2020).

TABLE 1
Herbaceous oil-bearing plants

| | Common | | | | | Oil | |
|---|---|---|---|---|---|---|---|
| No. | name | Species | Genus | Familia | Main producing area | content | References |
| 1 | Soybean | Glycine max (Linn.) Merr. | Glycine | leguminosae | China, the United States, | 18-24% | (Li et al., 2018) |
| | | | | sp. | Brazil et al. | | |
| 2 | Rape | Brassica napus L. | Brassica | Brassicaceae | All over the world | 37.5-46.3% | (Zhao et al., 2005; Ishaq |
| | | | | | | | et al., 2017) |
| 3 | Sunflower | Helianthus annuus | Helianthus | Compositae | All over the world | 46-50% | (Rauf et al., 2017) |
| 4 | Peanut | Arachis hypogaea L. | Arachis | leguminosae | Asia, Africa, America, et al. | 46-57% | (Wang X, et al., 2018) |
| | | | | sp. | | | |
| 5 | Cotton | Gossypium spp | Gossypium | Malvaceae | China, the United States, | 15-40% | (Shang et al., 2017) |
| | | | | | India, Uzbekistan, Egypt, | | |
| | | | | | etc. | | |
| 6 | Corn | Zea mays L. | Zea | Gramineae | Tropical and temperate | 4.5-4.8% | (Wang et al., 2010) |
| | | | | | regions of the world | | |
| 7 | Sesame | Sesamum indicum | Sesamum | Pedaliaceae | Tropical and temperate | 43-61% | (Latif and Anwar, 2011) |
| | | | | | regions | | |
| 8 | Hemp | Cannabis sativa L. | Cannabis | Moraceae | All over the world | 25-35% | (Vonapartis et al., 2014) |
| | | subsp. sativa | | | | | |
| 9 | Grape | Vitis vinifera L. | Vitis | Vitaceae | All over the world | 10-20% | (Movahed and Ghavami, |
| | | | | | | | 2007) |
| 10 | Fiberflax | Linum usitatissimum L. | Linum | Unaceae | Mediterranean region, | 35-45% | (Martinchik et al., 2012) |
| | | | | | Euro-Asian Temperature | | |
| | | | | | Zone | | |
| 11 | Safflower | Carthamus tinctorius L. | Chelonopsis | Labiatae | China, Russia, Japan, | About 40% | (Toma et al., 2014) |
| | carthamus | | | | North Korea, et al. | | |
| 12 | Rice | O. sativa | Oryza | Poaceae | Almost everywhere, | 15-23% | (Ju and Vali, 2005) |
| | | | | | expect Antarctica. | | |
| 13 | Perilla | Perilla frutescens (L) | Perilla | Labiatae | India, Myanmar, Japan, | 40-50% | (Liao et al., 2018) |
| | | Britt. | | | Korea, Indonesia, | | |
| | | | | | Russia, et al. | | |

TABLE 2
Woody oil-bearing plants

| | Common | | | | | Oil | |
|---|---|---|---|---|---|---|---|
| No. | name | Species | Genus | Familia | Main producing area | content | References |
| 1 | Oil palm | Elaeis guineensis | Elaeis | Arecaceae | Tropical regions of Africa, | 50-55% | (Kasemsumran et al., 2012) |
| | | Jacq. | | | tropical regions of China, | | |
| | | | | | Taiwan, Hainan and Yunnan. | | |
| 2 | Coconut | Cocos nucifera | Cocos | Arecaceae | Asia, Africa and Latin | 65-74% | (Marina et al., 2009) |
| | | L. | | | America | | |
| 3 | Olive | Olea europaea L. | Olea | Oleaceae | Mediterranean coast | 31-56% | (Sun et al., 2017; Olmo- |
| | | | | | | | Garcia et al., 2018) |
| 4 | Tea-oil tree | Camellia oleifera | Camellia | Theaceae | From Yangtze River Valley | 47.0-59.5% | (Chen et al., 2011) |
| | | Abel | | | to Southern China | | |
| 5 | Walnut | Juglans regia L. | Juglans L. | Juglandaceae | Southeastem Europe, | 60-70% | (Ôzcan et al., 2010) |
| | | | | | Himalaya mountains, China | | |
| 6 | Peony | Paeonia | Paeonia | Paconiaceae | Henan, Sichuan, Tibet, | 27-33% | (Ning et al., 2016; Zhang |
| | | suffruticosa Andr | | | Guizhou, Yunnan of China | | et al., 2018) |
| 7 | Pecan | Carya | Carya | Juglandaceae | Anhui and Zhejiang, China | 60-70% | (Huang et al., 2016) |
| | | cathayensis | | | | | |
| | | Sarg. | | | | | |
| 8 | Hazelnut | Corylus | Corylus | Betulaceae | Temperate zone in Asia, | 50-75% | (Balta et al., 2006; |
| | | heterophylla | | | Europe and North America | | Miraliakbari and Shahidi, |
| | | Fisch. | | | | | 2008; Juhaimi et al., 2018) |
| 9 | Idesia | Idesia polycarpa | Idesia | Flacourtiaceae | Southwest China, North | 21.2-44.0% | (Zhu, 2010; Gong et al., |
| | | Maxim. | | | Korea, South Japan. | | 2012; Li R. J. et al., 2016) |
| 10 | Pine | Pinus | Pinus | Pinaceae | Brazil, coniferous forests, | 58-69% | (Ryan et al., 2006; Bao and |
| | | | | | et al. | | Guo, 2016) |
| 11 | Cocoa | Theobroma | Theobroma | Sterculiaceae | Narrower within 10° north- | 45-60% | (Servent et al., 2018) |
| | | cacao L | | | south latitude of the equator | | |
| 12 | Shiny-leaved | Xanthoceras | Xanthoceras | Sapindaceae | North and northeast China | 50-60% | (Cao, 2015) |
| | yellowhorn | sorbifolium | Bunge | | | | |
| | | Bunge | | | | | |
| 13 | Acer | Acer truncatum | Acer Linn. | Aceraceae | Northeast and north China, | 42-46% | (Zhang and Hou, 2010) |
| | truncatum | Bunge | | | Shaanxi, Sichuan, et al. | | |

Exemplary edible structuring agents include, but are not limited to, polysaccharides, oligosaccharides, proteins, hydrocolloids.

Examples of edible structuring agents that are usable as edible structuring agents include free fatty acids, waxes, monoglycerides, diglycerides and more [see for instance Daniel and Marangoni Journal of the American Oil Chemists' Society volume 89, pages 749-780 (2012)].

According to a specific embodiment, the edible structuring agent is glycerol monostearate.

According to a specific embodiment, the coating which surrounds/coats the core is about 5-50 μm, e.g., 10-40 μm thick such as determined by SEM.

According to a specific embodiment, the average size of the particles is 0.1-50 μm, e.g., 0.1-40 μm, 0.1-30 μm, 0.1-20 μm, 0.1-10 μm, 0.1-5 μm, 0.5-50 μm, 1-50 μm, 5-50 μm, 10-50 μm, 20-50 μm, 0.1-50 μm, 0.5-15 μm, 0.5-10 μm, 0.5-20 μm, 0.5-5 μm.

According to a specific embodiment, the microparticles consist essentially of the plant protein.

According to a specific embodiment, the particles comprise an edible polymer so as to result in composite particles.

According to a specific embodiment, the edible polymer is a polysaccharide. Polysaccharides are constructed from monomeric sugars that are linked together by O-glycosidic linkages.

The polysaccharide is selected biocompatible and can be purified from nature or synthetically synthesized. The latter approach allows including some modifications which will increase its bioavailability, safety, improved mechanical properties or co-formulation properties with the plant protein.

Non-limiting examples are provided infra.

Cellulose—Cellulose is a type of polysaccharide found abundantly in nature and is easily chemically modified, which provides many advantages. Cellulose forms the structural basis in plants, which makes it the most abundant renewable resource on the planet. As a biomaterial, cellulose has served as wound dressings and in the form of hydrogels for orthopedic applications. Favorable properties include high tensile strength and biocompatibility. Different means of enhancing its properties has been explored, such as phosphorylation or bacterial synthetization, which increase its bioactivity.

Chitin and Chitosan—Chitin serves as a major structural component of invertebrates, insects, and fungi. It is an extremely abundant biopolymer, right after cellulose. In its purest form, it is insoluble in water. Its structure is a highly linear and it is a highly crystalline polymer. The material chitosan can be found in a few fungi species, and is mainly produced through chitin deacetylation. Due to its high degree of crystallinity, the materials are extremely stable through hydrogen bonding. These materials contain no antigenic properties, which makes them biocompatible as well as eco-friendly.

Starch—Starch is an abundant polysaccharide that is found in the roots, seeds, and stems of various plants and crops. Starch is constructed of anhydroglucose units and subsequently comprises two different polymers: Amylose and amylopectin. While starch presents a few disadvantages, such as low mechanical strength and high hydrophilicity, it has demonstrated good biodegradability and cell seeding capabilities. Therefore, starch has excellent structural capabilities for biodegradability and biocompatibility. Starch is relatively easy to modify, which makes it suitable for chemical enhancements to improve upon its weaker qualities.

Pectin-Pectin is a carbohydrate material derived from plant walls, mainly as a citrus byproduct. Pectin has excellent gelation properties. It is also hydrophilic in nature with many functional capabilities. It can be divided into three main regions: Smooth, hairy, and branched. The gelling property as well as solubility is dependent upon the esterification of galacturonic acid residues. Because of its gel-forming abilities, it has been recommended for the use of delivery bioactive agents. Pectin is non-toxic, and high in fiber content, which has made it successful in the food industry.

Alginates-Alginates are an important polysaccharide and can be found in algae species and soil bacteria. Being one of the most biosynthesized materials, alginates are naturally hydrophilic and anionic. Alginates have an excellent ability to store and retain water, as well as stabilizing and gelation properties. Chelation properties also make alginates favorable in drug delivery systems or tissue regeneration.

Hyaluronic Acid-Hyaluronic acid is a natural linear polysaccharide found in the extracellular matrix of animals. This material is naturally biocompatible, biodegradable, and lacks immunogenicity. Its structural properties give it the ability to mediate cell signaling, provide wound repair, and declare matrix organization.

Also contemplated herein are derivatives of these polysaccharides such as in the case of cellulose, e.g., carboxymetyl cellulose, methyl cellulose or hydroxypropyl cellulose.

Other polysaccharides include but are not limited to dextrin, carageenan, gum Arabic, and xanthan gum.

According to a specific embodiment the polysaccharide is alginate.

Be it essentially composed of the edible plant protein or a composite material comprising the polysaccharide too, the particles are cross linked at least to some extent.

According to some embodiments, the cross-linking agent is edible.

According to some embodiments, at least one of the plant protein and edible polymer is cross-linked.

According to some embodiments, the plant protein is cross linked (e.g., enzymatically).

According to some embodiments, the edible polymer is cross linked (e.g., alginate and Ca⁺²).

According to some embodiments, the edible polymer and the plant protein are cross linked to one another, and/or each is cross-linked independently.

According to some embodiments, the particles comprise a cross-linking agent (e.g., an enzyme).

The particles are endowed with advantageous properties, such as supporting cell proliferation, differentiation, viability.

As used herein, “proliferation” refers to a process that results in an increase in the number of cells. It is characterized by a balance between cell division and cell loss through cell death or differentiation.

In some embodiments, proliferation is measured by any method known to one skilled in the art. In some embodiments, proliferation is measured through direct cell counts. In certain embodiments, proliferation is measured by a haemocytometer. In some embodiments, proliferation is measured by automated cell imaging. In certain embodiments, proliferation is measured by a Coulter counter.

In some embodiments, proliferation is measured by using viability stains. In certain embodiments, the stains used comprise trypan blue.

In some embodiments, proliferation is measured by the total DNA. In some embodiments, proliferation is measured by BrdU labelling. In some embodiments, proliferation is measured by metabolic measurements. In certain embodiments, proliferation is measured by using tetrazolium salts. In certain embodiments, proliferation is measured by ATP-coupled luminescence.

The particles also exhibit stability which is important for an intended use as an edible carrier, as manifested by a zeta potential of −30 to −70 for oleogels).

Also contemplated herein are methods of producing the particles of the invention.

Thus, according to an aspect of the invention there is provided a method of producing the edible particles, the method comprising particulating a composition comprising an edible plant protein and optionally an edible polymer under conditions which allow formation of edible microparticles which support proliferation of cells cultured thereon.

As used herein “particulating” refers to the process of forming particles as opposed to tubes/fibers or sheets.

As used herein “fiber” or “tube” refers to aspect ratio (L/D) of >3, 5 or 10.

According to a specific embodiment the particles are spherical or oval.

Methods of producing particles are well known in the art. Measures are taken to use edible reagents (e.g., cross-linking reagents), while maintaining the biological properties of the plant protein and optionally edible polymer in supporting cell culturing thereon.

Some embodiments for producing the particles of the present invention are described herein and typically (but not necessarily) include cross-linking.

According to some embodiments, particulating involves the cross linking at least one of said edible plant protein and said edible polymer.

Crosslinking of the edible polymer and/or the plant protein can be achieved, for example, by chemical reaction, the application of energy such as radiant energy (e.g. UV light or microwave energy), drying and/or heating and dye-mediated photo-oxidation; dehydrothermal treatment; enzymatic treatment or others.

According to an embodiment, the plant protein (e.g., chickpea extract (CPE)) is mixed with an edible polymer such as alginate in 2:1, 5:1, 10:1, 15:1, 5:3 ratio (M) to form an aqueous solution of same. The solution can be electrosprayed into a cross-linking solution (e.g., comprising a cross-linking agent for chemically cross-linking the alginate and/or the plant protein; for example, calcium in the case of alginate). Particles are thus formed and washed.

The concentration of the cross-linkers is a matter of choice dependent on the level of cross-linking desired. Possible cross linkers include, but are not limited to, tripolyphosphate (TPP), Genipin, calcium chloride (CaCl₂), Ferrum, glycation, citric acid, aglycones (such as genipin), proanthocyanidins [such as proanthocyanidin (PA), and epigallocatechin gallate (EGCG)].

According to a specific embodiment, the chemical cross-linker (which cross links the protein) is EGCG, a component extracted from green tea with numerous biological activities including an anti-inflammatory effect. According to a specific embodiment, the concentration of EGCG is 0.1-3% (w/v) or 0.5-1% w/v.

Alternatively, following crosslinking and electrospraying, the cell carriers can be further exposed to a cross-linking treatment that cross-links the protein in the particles. This can be achieved by any of enzymatic cross-linking, chemical cross-linking, or both.

Chemical cross-linking of proteins can be achieved by organic acid treatment, e.g., citric acid. Examples of other acids which can be used in cross-linking the protein portion include but are not limited to, tannic, malic, lactic and hydrochloric acid.

As used herein “cross-linking enzyme” is an enzyme that catalyzes the formation of covalent bonds between one or more polypeptides.

Exemplary cross-linking enzymes are selected from the group consisting of transglutaminase, sortase, subtilisin, tyrosinase, laccase, peroxidase, and lysyl oxidase.

As used herein, “transglutaminase” or “TG” refers to an enzyme (R-glutamyl-peptide amine glutamyl transferase) that catalyzes the formation of a peptide (amide) bond between gamma.-carboxyamide groups and various primary amines, classified as EC 2,3.2,13. Transglutaminases catalyze the formation of covalent bonds between polypeptides, thereby cross-linked polypeptides. Cross-linking enzymes such as transglutaminase are used in the food industry to improve texture of some food products such as dairy, meat and cereal products. It can be isolated from a bacterial source, a fungus, a mold, a fish, a mammal, or a plant. According to a specific embodiment, the TG concentration is 20-200 units/gr protein.

Alternatively, only the protein portion of the particles is cross-linked by any of enzymatic cross-linking, chemical cross-linking, or both e.g., citric acid +/−TG).

According to an embodiment, cross-linked particles are generated in a mold, so as to provide a desired size and shape of the formed particles. For example, the plant protein solution (e.g., CPE) is stirred to remove any undissolved residues. The solution is then mixed with the edible polymer (e.g., alginate) and a cross linker (e.g., TG) to cross-link the protein. The solution is then subjected to a chemical cross-linking to cross-link the edible polymer (e.g., alginate-CaCl₂), molded (e.g., using a 3D-printed mold having a desired geometry and pattern).

Besides, or in addition to, cross-linking or gel-formation, the microparticles can be formed by any other methodology or technique known in the art for particles formation.

In some embodiments, microparticles are produced by spraying (e.g., electrospraying) an aqueous solution of the plant protein extract into a water-miscible organic solvent (e.g., an alcoholic solvent or solution).

In exemplary embodiments, the microparticles are produced by electrospraying an aqueous solution of the plant protein extract into an alcohol solution (e.g., ethanol).

According to a specific embodiment, the aqueous solution of the plant protein comprises a volatile water-miscible organic solvent.

According to a specific embodiment, the organic solvent is hexafluoroisopropanol.

For example, the plant protein extract is mixed with hexafluoroisopropanol (HFIP) and water and then electrosprayed into an ethanol solution. Following incubation, the HFTP is evaporated overnight, and the particles are washed.

Alternatively, microparticles can be produced by invention emulsion techniques, solvent-removal technique, molding, and any other methods known in the art, preferably such methods that can be successfully executed using GRAS or food contact substances.

According to some embodiments, the resultant particles are in a form of a hydrogel (e.g., hydrogel particles).

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of natural and/or synthetic polymeric chains, within the liquid dispersing medium.

Hereinthroughout, the phrases “aqueous solution” and “aqueous medium” are used interchangeably.

As used herein, the phrase “fibrous network” refers to a set of connections formed between a plurality of the protein.

By “hydrogel particles” it is therefore meant a plurality of discrete particles, each being discrete mass in a form of a hydrogel as defined herein.

According to a specific embodiment, the particles are subjected to lyophilization.

Accordingly, there is provided a preparation comprising lyophilized particles.

The term “lyophilized” or “freeze-dried” includes a state of a substance that has been subjected to a drying procedure such as lyophilization, where at least 50% of moisture has been removed.

The ability to promote cell viability, proliferation and even differentiation can be harnessed towards the use of the particles as described herein as cell carriers in suspension cultures.

Thus, according to an aspect of the invention there is provided a method of culturing, comprising culturing the composite which comprises microparticles as described herein and cells seeded thereon under conditions which allow differentiation and/or proliferation of said cells on the microparticles.

As used herein “cells” refer to non-human cells typically from an edible animal.

As used herein “animal cells” refers to any non-human cell (e.g., mammals, avian, insect, fish).

The cells can be wild-type cells or genetically modified cells (e.g., transgenic, genome edited).

According to a specific embodiment, the cells are terminally differentiated.

According to a specific embodiment, the cells are partially differentiated (e.g., blood-derived mesenchymal precursor cells, neural progenitor cells, multipotent adult progenitor cells, mesodermal progenitor cells, spinal cord progenitor cells).

According to a specific embodiment, the cells are stem cells (e.g., neural stem cells, multipotent stem cells from subventricular forebrain region, ependymal-derived neural stem cells, hematopoietic stem cells, liver-derived hematopoietic stem, marrow-derived stem cell, adipose-derived stem cells, islet-cells producing stem cells, pancreatic-derived pluripotent islet-producing stem cells, mesenchymal stem cells).

According to a specific embodiment, the cells are stem cells selected from the group consisting of mesenchymal stem cells and embryonic stem cells.

According to a specific embodiment, the cells are mesenchymal stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus, and cells originating from an unfertilized ova which are stimulated by parthenogenesis (parthenotes).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Hematopoietic stem cells, which may also refer to as adult tissue stem cells, include stem cells obtained from blood or bone marrow tissue of an individual at any age or from cord blood of a newborn individual.

Placental and cord blood stem cells may also be referred to as “young stem cells”.

Mesenchymal stem cells are multipotent stromal cells that can differentiate into a variety of cell types, including osteoblasts (bone cells), chondrocytes (cartilage cells), myocytes (muscle cells) and adipocytes (fat cells which give rise to marrow adipose tissue). The term encompasses multipotent cells derived from the marrow as well as other non-marrow tissues, such as placenta, umbilical cord blood, adipose tissue, adult muscle, corneal stroma or the dental pulp of deciduous baby teeth. The cells do not have the capacity to reconstitute an entire organ.

According to a specific embodiment, the cells are selected from the group consisting of erythrocytes, adipocytes, fibroblasts and muscle cells.

According to a specific embodiment, the microcarriers do not allow sufficient fusion to form cell fibers (i.e., muscle fibers).

According to a specific embodiment, the cells are of domesticated animals, such as listed below (e.g., duck, chicken).

According to a specific embodiment, the cells are produced by in vitro expansion e.g., stem cell expansion.

According to a specific embodiment, the cells are produced by in vitro differentiation.

Methods of cell differentiation are well known in the art.

Generally, these methods are based on suspension cultures using microcarriers.

To produce large amount of cells the use of bioreactors is contemplated.

As used herein “bioreactor” refers to a vessel, device or system designed to grow cells. Such bioreactors are described by Popovic et al. Biotechnology—Bioreactoes and Cultivation Systems for Cell and Tissue Culture—M. K. Popovic, Ralf Portner Encyclopedia of Life Support Systems (EOLSS) and further hereinbelow and in the Examples section which follows.

Selection of culture apparatus for production is based on the scale. Large-scale production preferably involves the use of dedicated devices. Continuous cell culture systems are reviewed in Furey (2000) Genetic Eng. News 20:10. Suitable bioreactors which can be used according to the present teachings include, but are not limited to, packed-bed bioreactors, fluidized-bed bioreactors, simulated microgravity bioreactors such as high aspect ratio vessel bioreactors, and slow turning lateral vessel bioreactors.

Typical bioreactors utilize a chamber filled with media, such as DMEM supplemented with serum/serum-replacement and/or growth factors and vented to ensure that there is a zero-head space in the reactor chamber. In some embodiments, the reactor chamber is then incubated at 37° C. and the media pumped through a media gas exchange module having its gas exchange tubing filled with a gas mixture as known to those of skills in the art.

In some embodiments, the bioreactor cell culture system is scalable for commercial production of viable cells. In some embodiments, the bioreactors of the present invention have been optimized for the expansion of stem cells. The derived cells may be initially cultured from frozen stocks in a tissue culture flask, trypsinized, and seeded onto the particles of the present invention. In some embodiments, the seeded particles are then introduced into the bioreactor. Typically, the cells and/or seeded carriers are introduced into the bioreactor chamber through a sampling port. In some embodiments, the vessel is slowly rotated without media flow for a few hours to allow an opportunity for the cells to efficiently seed the particles (carriers). In a suspension culture, cells may readily attach to the particles at rotational speeds between 1-7 rpm. After 24 hours, the media flow may be initiated and a sample of the media in the culture chamber may be taken and the number of unattached cells counted to assess the seeding efficiency to the particles.

In some embodiments, the compositions and methods can comprise a basal salt nutrient solution. A basal salt nutrient solution refers to a mixture of salts that provide cells with water and certain bulk inorganic ions essential for normal cell metabolism, maintain intra- and extra-cellular osmotic balance, provide a carbohydrate as an energy source, and provide a buffering system to maintain the medium within the physiological pH range. For example, basal salt nutrient solutions may include, but are not limited to, Dulbecco's Modified Eagle's Medium (DMEM), Minimal Essential Medium (MEM), Basal Medium Eagle (BME), RPM1 1640, Hams F-10, Ham's F-12, (beta-Minimal Essential Medium (beta-MEM), Glasgow's Minimal Essential Medium (G-MEM), and Iscove's Modified Dulbecco's Medium, and mixtures thereof.

Since the particles are edible there is no need to dissociate the cells from the particles once confluence is achieved.

Thus, according to some embodiments of the invention retrieving said cells from the culture is achieved without discarding the microparticles.

Regardless of the method employed, one the particles are at hand they can be used in the food/feed/beverage industry to produce comestible products, e.g., meat, or meat-like products.

Thus, the edible particles can be formed and/or combined with other molecules/substances with high nutritional value or providing improved texture or adding flavor to the final product. They can also be used for the simultaneous culturing of several cell types with relevance to meat, thus allowing for the engineering of novel foods. For example, bovine and fish cells could be cultured simultaneously.

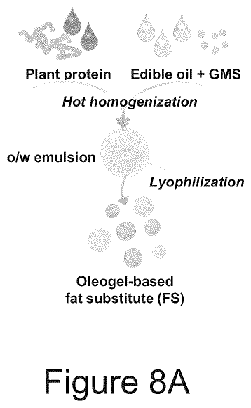

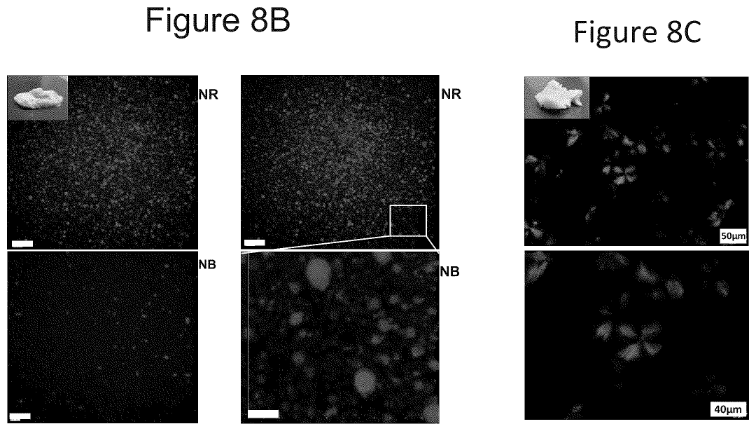

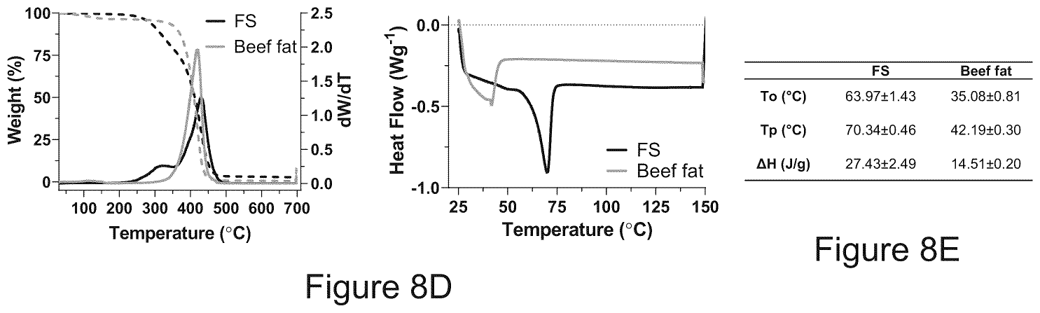

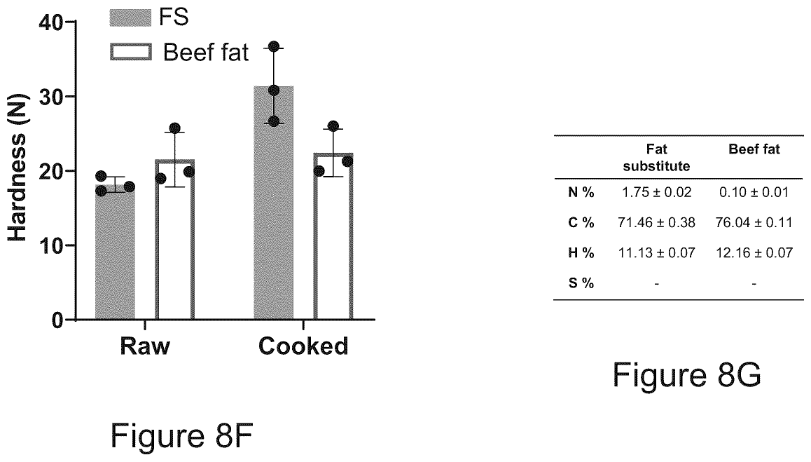

It will be appreciated that the particles can be used per se (i.e., without cells cultured thereon). This embodiment is of value, since the particles being made of plant protein, optionally as oleogel, are highly nutritious. Oleogel particles may impart foods with beef fat-like characteristics as demonstrated in FIGS. 8A-G by their degradation behaviour over the indicated temperatures; scanning calorimetry (DSC) thermogram (10° C. min⁻¹); Hardness (N) values; and elemental analysis (C, H, N, and S).

Thus, according to an aspect there is provided a food, feed or beverage comprising the composition of composite as described herein.

Also provided is a method of producing food, the method comprising fabricating food comprising the composite or composition as described herein.

According to some embodiments, the fabricating comprises molding, extruding, electrospinning and/or printing.

According to other embodiments, the particles are combined with other food ingredients to form consumables such as minced meat, burger, nugget, sausage or patty.

It is thus within the scope of the invention wherein the food, feed or beverage additionally comprising acidity regulators, anticaking agents, antifoaming agents, natural and other antioxidants, bulking agents, food coloring agents, color retention agents, emulsifiers, flavors, flavor enhancers, flour treatment agents, glazing agents, humectants, tracer gas, preservatives, probiotic microorganisms, stabilizers, sweeteners, thickeners and any mixtures thereof.

In other embodiment of the present invention, the food, feed or beverage product is a consumable, edible item having the final organoleptic properties of a meat product, and especially product(s) selected from the group consisting of Beef, Beef heart, Beef liver, Beef tongue, Bone soup from allowable meats, Buffalo, Bison, Calf liver, Caribou, Goat, Ham, Horse, Kangaroo, Lamb, Marrow soup, Moose, Mutton, Opossum, Organ Meats, Pork, Bacon, Rabbit, Snake, Squirrel, Sweetbreads, Tripe, Turtle, Veal, Venison, Chicken, Chicken Liver, Cornish Game Hen, Duck, Duck Liver, Emu, Gizzards, Goose, Goose Liver, Grouse, Guinea Hen, Liver, Ostrich, Partridge, Pheasant, Quail, Squab, and Turkey.

It is expected that during the life of a patent maturing from this application many relevant edible components (e.g., edible polymers, cross-linking agents, structuring agents, oils, etc.) will be developed and the scope of these terms is intended to include all such new technologies a priori.

As used herein the term “about” refers to ±5 or 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Methods

### Chickpea Protein Extract (CPE) Preparation—

Extraction was performed according to a protocol by Glusac J, Isaschar-Ovdat S, Fishman A. Transglutaminase modifies the physical stability and digestibility of chickpea protein-stabilized oil-in-water emulsions. Food Chem. 2020; 315:126301. Briefly, fresh chickpea seeds were grounded and the obtained chickpea powder was mixed with distilled water at a 1:10 ratio (w/v), adjusted to pH 9.0 using 1.0M NaOH and stirred at 500 rpm for 90 min at room temperature (20-22° C.). The suspension was then centrifuged (Thermo Scientific™ Sorvall™ LYNX 4000, Thermo Fisher Scientific, Waltham, MA USA) at 4500×g for 20 min at 4° C. to collect the supernatant. The resulting pellet was resuspended in distilled water at a ratio of 1:5 (w/v), adjusted to pH 9.0, stirred for an additional 90 min, followed by centrifugation (4500×g, 25 min, 4° C.). Both supernatants were pooled and adjusted to pH 4.6 using 0.1M HCl to precipitate the protein. The protein precipitate was recovered by centrifugation, dissolved in distilled water and the pH was raised to 7.0. The samples were dialyzed against water and freeze-dried.

### CPE Effect on Mammalian Cell Culture

Bovine umbilical cord-derived mesenchymal stem cells (bMSC) were seeded on chickpea (CP) extract (CPE)-coated non-adherent tissue culture (TC) plates at a density of 5000 cells/cm². Following 3 days, cells attachment to the plates was verified and compared to regular TC plates, non-adherence TC plates, and gelatin-coated TC plates using inverted bright field microscopy. Additionally, cell morphology under the different conditions was compared using phalloidin-DAPI staining. The expression of MSC typical markers on the surface of cells cultured under the different conditions was compared using FACS analyses and antibodies for CD29 and CD44 (anti CD29: 303008, Biolegend, anti CD44: 103011, Biolegend).

To address CPE possible cytotoxic effect, bMSC or sheep fibroblasts were seeded on TC plates at a density of 2,500 cells/cm² and 10,000 cells/cm. One day later, the culture medium was supplemented with increasing concentrations of CPE, up to 1 mg/ml. Cell viability was followed along 4 days using Alamar blue and compared to untreated cells and cells treated with cytotoxic Triton X100 at 0.05%.

A fourth method—Beads based on cellulose/alginate were chemically crosslinked using CaCl₂). Encapsulation of CP-carrier in accordance with the present invention was performed as follows:
1. In the first step the following ingredients were taken: CP protein ground to form a homogenous powder with a mean particle size of less than 1 micron.
2. A solution containing Sodium Alginate and water.
   It is to be noted that in some embodiments, a surfactant can be added at the milling stage (step 1, above), or to the alginate solution prepared in this step.
3. Powder (a) and Solution (b) were mixed together to form a homogenous suspension, which was fed through a dispenser, installed above a 1.5% CaCl₂) solution in water. The spherical droplets, upon entering the solution, gelled to form alginate matrix beadlets when retained in the solution for 10 to 30 minutes. The beadlets were collected by filtration and washed in 2.5% citric acid in water followed by a second wash in water. The beadlets were dried with a fluidized bed drying apparatus.

### Production of Chickpea-Alginate Cell Carriers

To produce CP-alginate cell carriers, 10% CPE solution in Tris HCl buffer (pH=10) was mixed with 2% aqueous sodium alginate solution (1:1) and electrosprayed into a crosslinking solution of 15 mg/ml CaCl₂) at pH=2. These carriers were washed with water, then with PBS, and finally with culture medium and seeded with cells.

Alternatively, following CaCl₂) crosslinking and electrospraying the cell carriers were treated with transglutaminase (TG, 100 U/gr CPE) for 2 hours in 15 mg/ml CaCl₂) at pH=2, or in sodium acetate buffer (50 mM, pH=4), or in MES buffer (50 mM, pH=6), or in PBS at pH=7.4. The cell carriers were then treated with citric acid 0.5M for 30 minutes and washed as previously described prior to seeding with cells.

A third method for producing CP-Alginate cell carriers, included electrospraying a CPE-alginate solution containing denatured CPE and no Calcium ions into a crosslinking solution of citric acid 0.1-2%, followed by washes as previously described and seeding with cells.

### Production of 100% Chickpea Cell Carriers

To produce CP cell carriers, a 13% CPE solution in 85% Hexafluoroisopropanol (HFIP) and 15% water was electrosprayed into a 96% ethanol solution. Following a 60 minute incubation, the HFIP was evaporated overnight in 70° C., and the cell carriers were washed as previously described and seeded with cells.

### Production of CPE-Coated Oleogel Microparticles

To produce CPE-coated oleogel particles, high shear homogenization was used. CPE solution was treated with transglutaminase (50 U/gr protein) for 2-3 hours at room temperature. Then, canola oil with glycerol monostearate (GMS) and CPE dispersion was heated to allow hot shear homogenization at temperatures above the GMS melting temperature (70° C.) and subsequently cooled to allow the formation of solid particles. Different formulations were used, as detailed in Table 3. Particle size distribution was determined using Mastersizer and Zetasizer 24 h after production and over 1 month of the storage at 4° C. Samples were 1000×diluted in 50 mM Tris-HCl pH=10 and zeta potential of the particles was calculated as well. Each measurement was the average of 10 measurements. To address their long-term stability, the microparticles were lyophilized and characterized using SEM (Phenom). Size distribution following redispersion was characterized as before.

TABLE 3
Formulations of oleogel microparticles.

| | Canola Oil | Glycerol monostearate | Protein | TG |
|---|---|---|---|---|
| No. | (%) | (%) | (%) | (50 U/g) |
| 1 | 75 | 25 | 0.1 | 50 |
| 2 | 75 | 25 | 0.5 | 50 |
| 3 | 75 | 25 | 1 | 50 |
| 4 | 75 | 25 | 1.5 | 50 |
| 5 | 75 | 25 | 2 | 50 |

GMS and additives concentrations are out of the oil content.
Protein concentration is out of the water content.

### Example 1

### CPE Effect on Mammalian Cell Culture

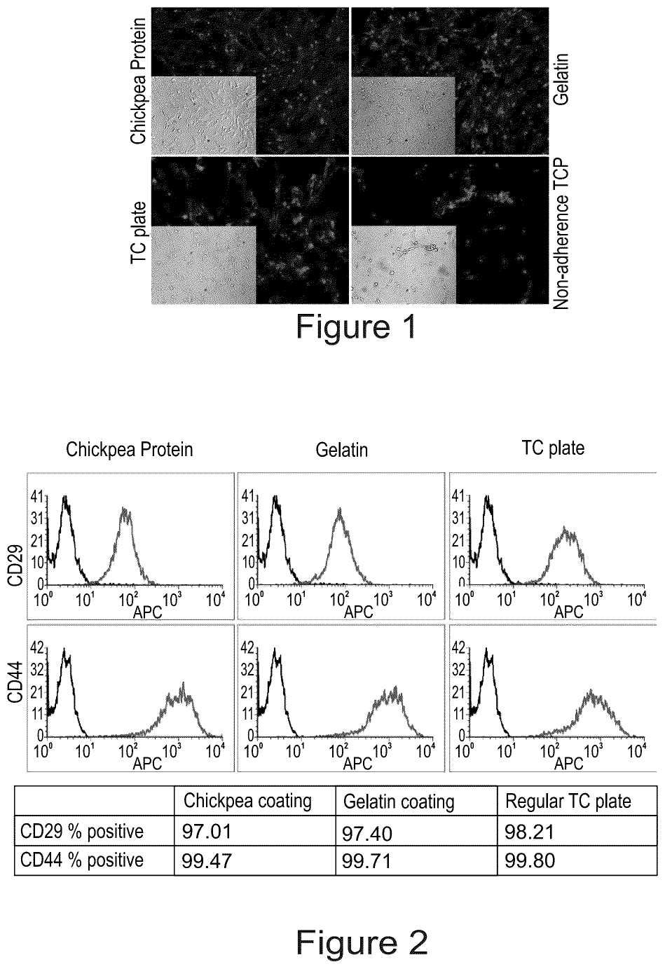

In order to develop edible cell carriers based on chickpea protein, the present inventors first established the feasibility to culture mammalian cells on CPE. The ability of bMSC to attach and proliferate on CPE-coated surfaces was verified through a comparison to regular TC plates, non-adherence TC plates, and gelatin-coated TC plates, thus revealing cell behavior similar to the gelatin coated ones (FIG. 1). Furthermore, the CPE coating did not affect the expression of the MSC typical markers CD29 and CD44 (FIG. 2).

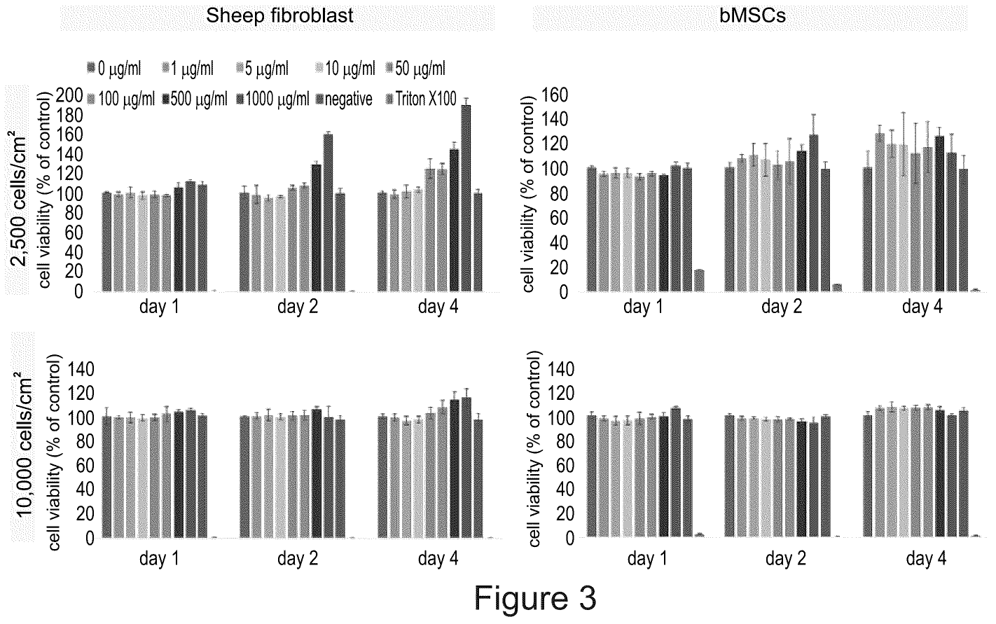

To ensure that chickpea protein has no cytotoxic effect, increasing concentrations of CPE were added to the cell medium, thus revealing no effect on cell viability along 4 days when the cells were seeded at 10,000 cells/cm². When the cells were seeded at 2,500 cells/cm², however, cell viability increased when treated with CPE starting from the 2^{nd} day of culture (FIG. 3).

### Example 2

### Preparation of Chickpea-Alginate Cell Carriers

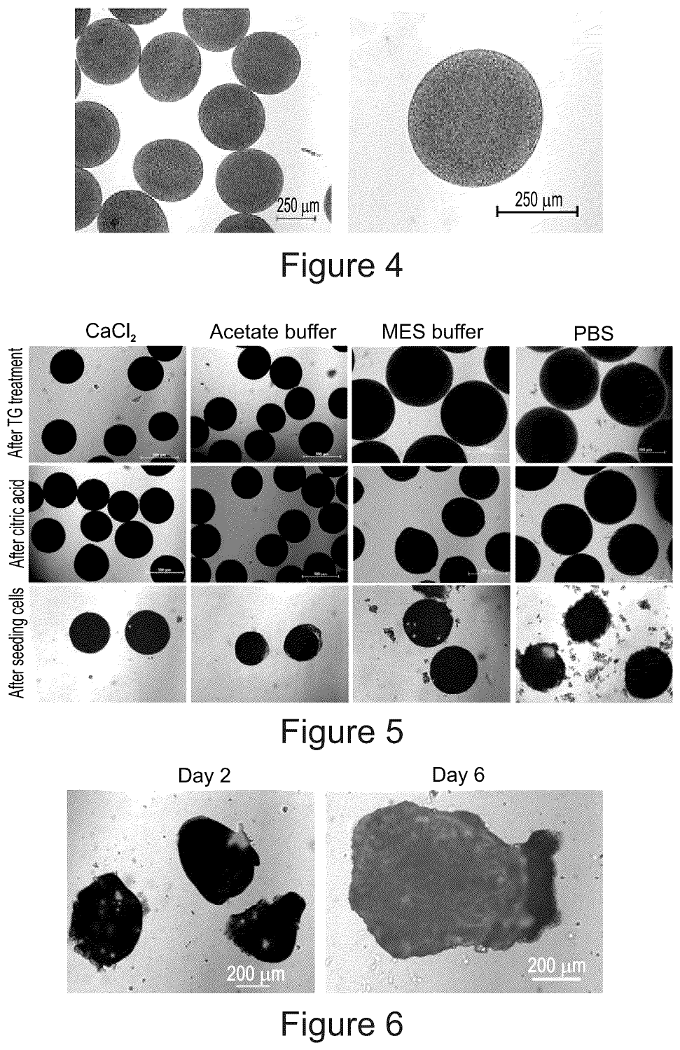

The first technological approach for the production of cell carriers from chickpea protein relied on the fast kinetics of alginate crosslinking in the presence of calcium ions to allow the instant formation of microparticles. The chickpea protein solution was mixed with alginate and the mixture was electrosprayed into a calcium crosslinking solution. As seen in FIG. 4, the produced microparticles were spherical and homogeneous, of around 400 micrometers in diameter.

Another type of chickpea-alginate cell carriers were produced by further using transglutaminase crosslinking in addition to citric acid treatment to stabilize the chickpea protein and chelate the calcium ions, thus solubilizing and removing most of the non-cross-linked alginate. In FIG. 5, cell carriers are shown following TG treatment in different buffers and following subsequent citric acid treatment. Furthermore, cell attachment at different rates to these cell carriers is demonstrated. The size of all the produced microcarriers was uniform for each preparation conditions and ranged between 350 to 700 micrometers, a suitable size for cell culturing.

The third type of chickpea-alginate cell carriers developed, was produced through the direct crosslinking using citric acid. The citric acid converts the sodium alginate into alginic acid, which solidifies, and also interferes with the structure of denatures CP so as to form particles. As shown in FIG. 6, these cell carriers were not spherical. Nevertheless, they supported cell attachment and proliferation, thus leading to a complete coverage of the carriers' surface with cells within 6 days of culture. Furthermore, when covered with cells, these carriers formed aggregates that may facilitate producing tight structures of cultured meat.

### Example 3

### Preparation of 100% Chickpea Protein Cell Carriers

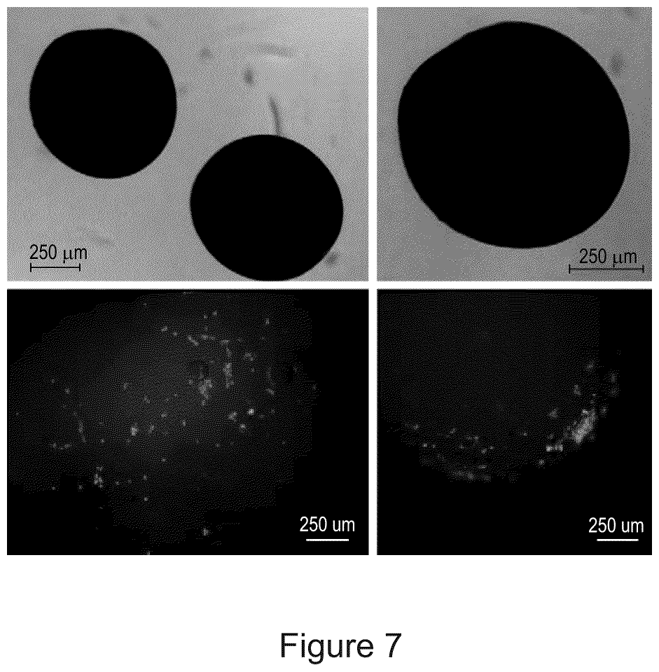

To develop chickpea protein cell carriers that do not rely on calcium-alginate crosslinking, the present inventors developed 100% chickpea protein-based cell carriers through the temporal use of hexafluoroisopropanol (HFIP), a water-miscible organic solvent that due to its high volatile nature, can be easily cleared from the produced carriers. CPE particles were formed upon electrospraying an HFIP-containing aqueous solution into an ethanol solution. The present results demonstrated the feasibility of this approach of production, with the generation of spherical shaped microcarriers of relevant size. The results are shown in FIG. 7.

### Example 4

### Development of CPE-Coated Oleogel Microparticles

Oleogel-based fat substitute holds the potential to provide clean meat with textures that mimic animal-derived fat, but with improved nutritional values. Primarily, the present inventors took the approach of developing oleogel-based microparticles, employing high shear homogenization to produce solid lipid nano-micro dispersions using GMS as a structuring agent of canola oil oleogel with the addition of chickpea protein coating.

More specifically O/W emulsion-based template, followed by lyophilization, was used as a method for oleogel production. The chickpea protein dispersions (4.2 5%, w/w) in distilled water were stirred overnight at 4° C. The oleogels were prepared using canola oil (20% o/w, w/w) followed by the addition of a 20% (w/w) glycerol monostearate (GMS) structuring agent (DMS 0091, Palsgaard®, Denmark). Emulsions were obtained by hot shear homogenization (800 rpm/30 sec, 16000 rpm/3 min) at temperatures above the melting temperature of the lipid (70° C.). After homogenization, emulsions were gradually cooled down (4 h/RT, 4 h/4° C., −80° C./overnight) and lyophilized. Oleogel was characterized and compared to commercial beef fat (purchased from a local butcher shop). Results are shown in FIGS. 8A-G.

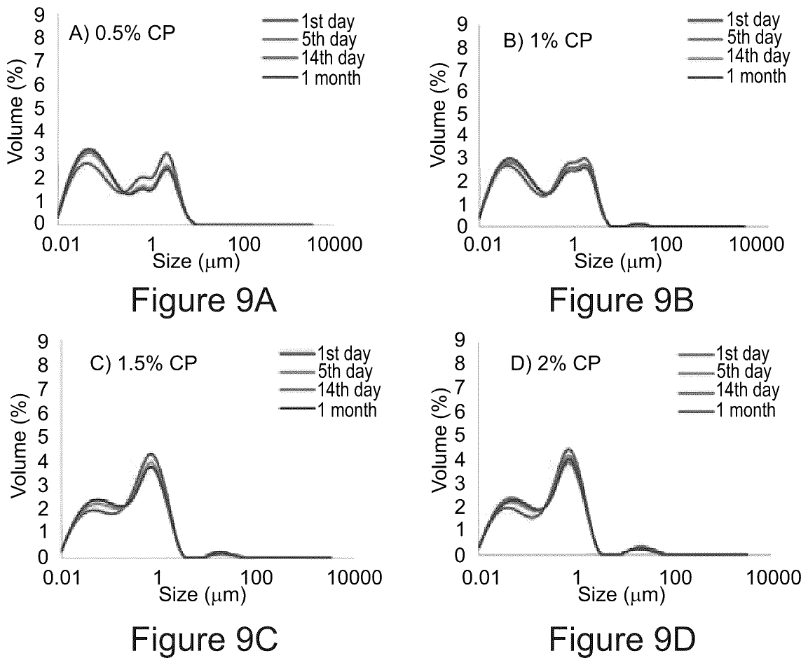

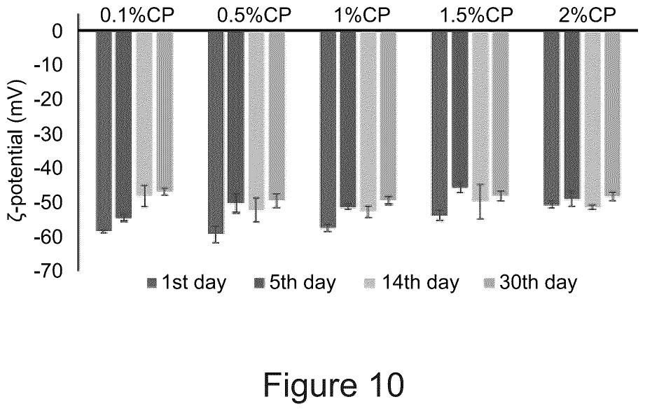

The produced oleogel microparticles were also characterized by a bimodal size distribution, which remained stable for an entire month (FIGS. 9A-D). Zeta potential was calculated, showing strongly net negative charge on droplet in the range from −50 to −62 mV over the storage period with a small decline over the storage time (FIG. 10).

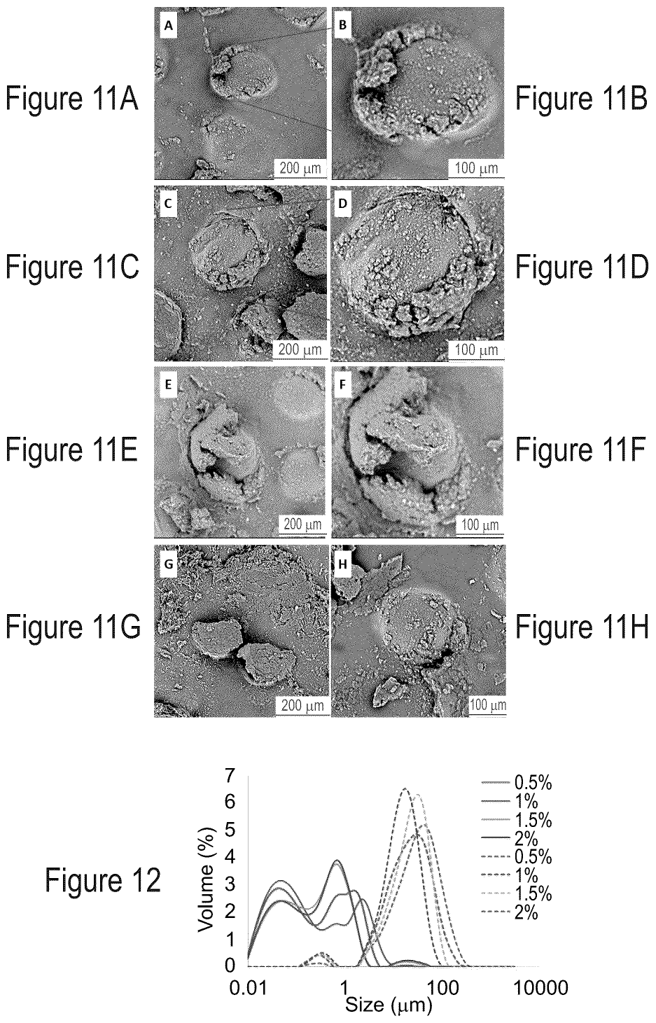

To preserve the oleogel microparticles for a longer-term, their lyophilization was addressed. Following lyophilization, the oleogel microparticles were characterized using SEM. From the SEM micrographs (FIGS. 11A-H) it can be observed that the surface of the oleogel microparticles is covered with a layer of smaller CP particles that grows into a thicker, more homogenous layer when increasing the CP concentration up to 1.5%. The lyophilized emulsions formulated using 0.5-2% CP showed the ability to re-disperse. When analyzing particle size following re-dispersion, however, the size distribution of the reconstituted emulsions showed a shift towards larger particle sizes (FIG. 12).

Redispersed emulsions showed monomodal distribution with the mean particle size between 20-60 m. This increase in particle size is probably due to particle aggregation during the lyophilization, which occurred less in the presence of the higher protein concentration. Oleogel particles with 0.1% CP were not redispersable, which could be attributed to the formation of larger fat droplets during freeze-drying. Thus, the protein might have a function as a coating agent of the oleogel particles and, therefore, preventing aggregation during the freeze-drying.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition-of-matter comprising a plurality of edible microparticles at least a portion of said edible microparticles comprises a plant protein.

2. The composition of claim 1, wherein said plant protein is in a plant protein extract, optionally wherein said plant of said plant protein is a legume.

3. The composition-of-matter of claim 2, wherein said legume is chickpea.

4. The composition-of-matter of claim 1, wherein at least a portion of said microparticles are essentially irregular in shape or at least a portion of said microparticles are essentially spherical.

5. The composition-of-matter of claim 1, wherein said microparticles comprise an oleogel core.

6. The composition-of-matter of claim 5, wherein said microparticles comprise said oleogel core and a continuous or non-continuous coating comprising said plant protein or wherein said oleogel core comprises an edible oil and an edible structuring agent.

7. The composition-of-matter of claim 6, wherein said edible structuring agent comprises glycerol monostearate.

8. The composition-of-matter of claim 6, wherein said coating is about 10-40 μm thick.

9. The composition-of-matter of claim 6, wherein said plant protein is cross-linked using an enzyme.

10. The composition-of-matter of claim 1, further comprising an edible polymer optionally wherein said edible polymer is selected from the group consisting of an alginate, a chitosan and a dextrin.

11. The composition-of-matter of claim 10, wherein said edible polymer is cross-linked, and wherein a cross-linking agent for cross linking said edible polymer is edible optionally wherein said edible polymer is alginate and said cross-linking agent for cross linking said edible polymer is Ca⁺².

12. The composition-of-matter of claim 1, wherein said plant protein is cross-linked: optionally cross linking is by using and enzyme and optionally wherein said enzyme is transglutaminase (TG); or cross linking is by using a chemical and optionally said chemical is an organic acid.

13. The composition-of-matter of claim 12, wherein said organic acid is citric acid or wherein said chemical is epigallocatechin gallate (EGCG).

14. The composition-of-matter of claim 10, wherein at least one of said plant protein and edible polymer is cross-linked.

15. The composition-of-matter of claim 1, wherein said microparticles consist essentially of said plant protein extract.

16. The composition-of-matter of claim 15, wherein said microparticles are produced by electrospraying an aqueous solution of said plant protein extract into an alcohol solution.

17. The composition-of-matter of claim 16, wherein said aqueous solution of said plant protein comprises a volatile water-miscible organic solvent and optionally said organic solvent is ethanol.

18. The composition-of-matter of claim 1, wherein an average size of said microparticles is 50-600 μm and/or wherein an average zeta potential of said microparticles is −30 to −70 mV when comprising an oleogel core.

19. A method of producing the edible particles, the method comprising particulating a composition comprising an edible plant protein and optionally an edible polymer under conditions which allow formation of edible microparticles which support proliferation of cells cultured thereon.

20. A composite comprising the composition-of-matter of claim 1 and cells adhered to said microparticles and optionally wherein said cells comprise terminally differentiated cells.
